(19) United States
(12) Patent Application Publication
(10) Pub. No.: US 2010/0174300 A1
(43) Pub. Date: Jul. 8, 2010

(54) SUTURELESS VESSEL ANASTOMOSIS METHOD AND APPARATUS

(76) Inventor: Phillip Nicolas Blondeel, Merelbeke (BE)

Correspondence Address: GIFFORD, KRASS, SPRINKLE,ANDERSON & CITKOWSKI, P.C, PO BOX 7021, TROY, MI, 48007-7021, US

(21) Appl. No.: 12/664,756

(22) PCT Filed: Jun. 20, 2008
(86) PCT No.: PCT/IB08/01628

§ 371 Date: Dec. 15, 2009

Related U.S. Application Data
(63) Continuation-in-part of application No. 11/766,840, filed on Jun. 22, 2007.

(51) Int. Cl. A61B 17/11

(52) U.S. Cl. 606/154; 606/155

(57) ABSTRACT

A method for performing end-to-end vessel anastomosis includes providing a vessel support including a vessel receiving portion and a handle portion extending therefrom. The vessel receiving portion is an annulus. The vessel receiving portion is positioned around the end of a first vessel and the end of the first vessel is everted about the outer diameter of the vessel receiving portion such that the inner surface of the vessel is directed outwardly. The end of the second vessel is positioned over the everted end of the first vessel such that the inner surface of the second vessel is disposed against the inner surface of the first vessel. The ends of the vessel are secured either with a suture or a surgical adhesive. The handle portion of the vessel support may be removed.

### REFERENCE TO RELATED APPLICATIONS

This application is a continuation-in-part and claims priority of U.S. patent application Ser. No. 11/766,840 filed Jun. 22, 2007, the content of which is incorporated herein by reference in its entirety.

### FIELD OF THE INVENTION

The present invention relates generally to methods and apparatus for performing anastomoses of hollow organs such as blood vessels.

### BACKGROUND OF THE INVENTION

Many surgical procedures require attachment of hollow organs to each other, generally referred to as anastomosis. Most often, this procedure is required with blood vessels, referred to as vessel anastomosis. FIGS. 1A-1C illustrate a typical procedure for end-to-end anastomosis of a pair of blood vessels. The vessels are butted end-to-end and stitched together with small separate or running sutures. This approach to surgical interconnection may be used with both large and small vessels, with the number and size of sutures depending on the size of the vessel. A disadvantage to using sutures for anastomosis is that the success and patency rate of the procedure is directly related to the skills and dexterity of the surgeon. Anastomosis of small vessels is both difficult and time consuming.

The procedure illustrated in FIGS. 1A-1C also has the disadvantage that it introduces a foreign material, the suture, into the blood vessel, which may eventually lead to thrombosis (FIG. 1C).

In order to function properly, blood vessels need to be attached to one another such that they are strong and leakproof. Additionally, arteries are subject to a pressure wave of blood traveling down the vessel that expands and relaxes the vessel.

Human arteries include three layers. The outer layer, or adventitia, is fibrous and provides the support structure for the vessel. The middle layer, or media, consists of muscular fibers as well as collagen and elastin connective proteins. The inner layer, or intima, is a specialized mucosa that serves as a lining of the vessel. The open inner passage of the vessel is referred to as the lumen. If the intima layer of a blood vessel is damaged, the middle layer, or media, is exposed to blood. A repair function is stimulated leading to the formation of blood clots due to the contact of the blood with the exposed collagen of the media.

There have been numerous attempts to provide methods and apparatus that improve on vessel anastomosis using sutures. Examples include “welding” the vessel ends to each other using a heat source such as a laser as well as attachment using a wide variety of mechanical attachment devices. The use of a laser or other heating device is disadvantageous in that it requires manipulation of the heating device in a confined space. This is especially true with microsurgery and microvessel anastomosis. Reattachment using mechanical connectors has the disadvantage that it introduces foreign matter into the body. Also, many connector devices include gripping or piercing elements that damage the intima of a blood vessel, leading to the potential for blood clots.

In light of the above, there is an ongoing need for improved methods and apparatus for anastomoses of hollow organs including end-to-end vessel anastomoses.

### SUMMARY OF THE INVENTION

Embodiments of the present invention provide improved apparatus and methods for anastomoses of hollow organs such as blood and other vessels. A method according to one embodiment of the present invention is for end-to-end vessel anastomosis. A vessel support is provided that includes a vessel receiving portion and a handle portion extending therefrom. The vessel-receiving portion comprises an interrupted annulus having an inner diameter and an outer diameter. The handle portion is removable from the vessel-receiving portion. The vessel-receiving portion of the vessel support is positioned around the end of a first vessel such that the end of the first vessel is disposed through the inner diameter of the vessel-receiving portion. The end of the first vessel is everted around the outer diameter of the annulus of the vessel-receiving portion such that an inner surface of the vessel is directed outwardly. The end of a second vessel is positioned over the inverted end of the first vessel such that the inner surface of the second vessel is disposed against the inner surface of the first vessel. An internal adhesive is provided and is used to adhere the ends of the vessels together. The adhering step does not require the application of heat or radiant energy. The handle portion is then removed from the vessel support. In some versions, the handle portion is interconnected with the vessel-receiving portion by a frangible connection. In some versions, at least the vessel-receiving portion of the vessel support is formed of a resorbable material. In further versions, the annulus has an outer surface that is textured in order to improve adherence of the vessel thereto. In further versions, the annulus may have outwardly extending gripping elements, such as spikes, to engage the outer layer or adventitia of the vessel when it is everted thereon. In yet a further version, the annulus has a circumferential groove or depression defined in the outer surface to allow use with a single suture.

The size and shape of the vessel support may vary depending on the application. For microvessel anastomosis, a vessel support according to the present invention may have a vessel-receiving portion with an inner diameter in the range of 0.4 to 5 mm and a wall thickness of less than 0.5 mm. The annulus may have a longitudinal end-to-end length in the range of 1.6 to 10 mm. Exemplary materials for forming the overall vessel support or at least the vessel receiving portion include non-resorbable and biocompatible materials such as silicone, titanium or any other substance that is hard enough to resist the strain and is biocompatible. Exemplary materials also include resorbable and biocompatible such as hyaluronic acid in solid form or other biocompatible polymers. Other exemplary dimensions and materials may be used.

In an alternative embodiment of the method for performing end to end vessel anastomosis is also provided. A vessel support is provides which includes a vessel receiving portion and a handle portion extending therefrom. The vessel receiving portion is generally annular and has an inner diameter and an outer diameter. The generally annular vessel receiving portion has an outer surface with a recessed region defined thereon. The vessel receiving portion of the vessel support is positioned around the end of a first vessel such that the end of the first vessel is disposed thru the inner diameter. The end of the first vessel is everted around the outer diameter of the annulus of the vessel receiving portion such that an inner surface of the vessel is directed outwardly. The end of a second vessel is positioned over the everted end of the first vessel such that an inner surface of the second vessel is disposed against the inner surface of the first vessel. A suture is positioned around an outer surface of the end of the second vessel after the end of the second vessel is positioned over the everted end of the first vessel. The suture is generally aligned with the recessed region on the outer surface of the vessel receiving portion.

The outer surface of the vessel receiving portion may have first and second raised elements defined thereon and extending upwardly from the outer surface, with the recessed region being defined as the region between the raised elements. At least one of the raised elements may be a circumferential ring. At least one of the raised elements may be a plurality of gripping elements extending upwardly from the outer surface of the vessel receiving portion, the gripping elements being spaced apart circumferentially around the vessel receiving portion.

Further embodiments of the present invention provide a blood vessel support for performing an end-to-end vessel anastomosis. The support includes a vessel-receiving portion formed of a biocompatible material. The vessel-receiving portion has a generally annular shape with an outer surface and an inner surface. The inner surface defines an inner diameter of the vessel-receiving portion. The vessel-receiving portion has a wall thickness defined between the inner and outer surfaces. The generally annular vessel-receiving portion is interrupted so as to define a pair of spaced apart ends. A handle portion is removably interconnected with the vessel-receiving portion and extends therefrom. The inner diameter of the vessel-receiving portion is in the range of 0.3 to 5 mm and the wall thickness of the vessel-receiving portion is less than 0.5 mm.

The present invention also provides an alternative blood vessel support for performing an end to end vessel anastomosis. The blood vessel support has a vessel receiving portion formed of a biocompatible material, the vessel receiving portion having a generally annular shape with an outer surface and an inner surface. The inner surface defines an inner diameter of the vessel receiving portion. The vessel receiving portion has a wall thickness defined between the inner and outer surfaces. The outer surface has a first raised element and a second raised element defined thereon. Each raised element extends upwardly from the outer surface and also extends generally circumferentially around the vessel receiving portion. A generally circumferential recessed region is defined between the first and second raised elements. A handle portion is interconnected with the vessel receiving portion and extends therefrom.

One or both of the raised elements may be a circumferential ring. At least one of the raised elements may be a plurality of gripping elements extending upwardly from the outer surface of the vessel receiving portion, the gripping elements being spaced apart circumferentially around the vessel receiving portion. The vessel receiving portion may be said to have a leading edge and a trailing edge, and the gripping elements may be disposed adjacent the leading edge. The gripping elements may be spikes or hooks extending generally radially outwardly from the outer surface. The generally annular vessel receiving portion may be interrupted so as to define a pair of spaced apart ends with a gap therebetween. The inner diameter of the vessel receiving portion may be in the range of 0.3 to 5 mm and the wall thickness of the vessel receiving portion may be in the range of 50 to 400 microns. The vessel receiving portion may be formed of a resorbable material and the handle portion may be interconnected with the vessel receiving portion by frangible connection.

### BRIEF DESCRIPTION OF THE DRAWINGS

FIGS. 1A-1C are perspective views illustrating a pair of vessels being joined end to end using a traditional suturing approach to anastomosis; FIG. 1C illustrates an intraluminal thrombosis and total obstruction to intraluminal blood flow;

FIG. 2 is a perspective view of a first embodiment of a blood vessel support according to the present invention;

FIG. 2A is a perspective view of a second embodiment of a blood vessel support with a texture according to a further aspect of the present invention;

FIG. 3 is a perspective view of a third embodiment of a blood vessel support according to the present invention;

FIG. 4 is a perspective view of a fourth embodiment of a blood vessel support according to the present invention;

FIG. 5 is a perspective view of a fifth embodiment of a blood vessel support according to the present invention;

FIG. 6 is a perspective view of the blood vessel support of FIG. 5 with dimension markings;

FIG. 7 is a perspective view of a sixth embodiment of a blood vessel support according to the present invention;

FIG. 7a is a detailed view of a barbed hook;

FIG. 8 is a perspective view of the blood vessel support of FIG. 7 with dimension markings;

FIG. 9 is a perspective view of a seventh embodiment of a blood vessel support according to the present invention;

FIG. 10 is a perspective view of a pair of blood vessels to be joined end to end using an apparatus and anastomosis method according to the present invention;

FIG. 11 is a perspective view of the blood vessels of FIG. 10 after the end of one vessel is everted over the blood vessel support;

FIG. 12 is a perspective view of the blood vessels of FIGS. 10 and 11 with adhesive applied to the everted end;

FIG. 13 is a perspective view of the blood vessels of FIGS. 10-12 after the end of the second vessel has been positioned around the everted end of the first blood vessel and additional adhesive has been applied;

FIG. 14 is a perspective view of the blood vessels of FIGS. 10-13 with the handle of the blood vessel support being removed;

FIG. 15 is a perspective view of blood vessels joined using an alternative method and support making use of a suture to secure or reinforce the joint between the vessels; and

FIGS. 16A-16C are perspective views of an alternative anastomosis method according to the present invention for interconnecting the ends of larger vessels.

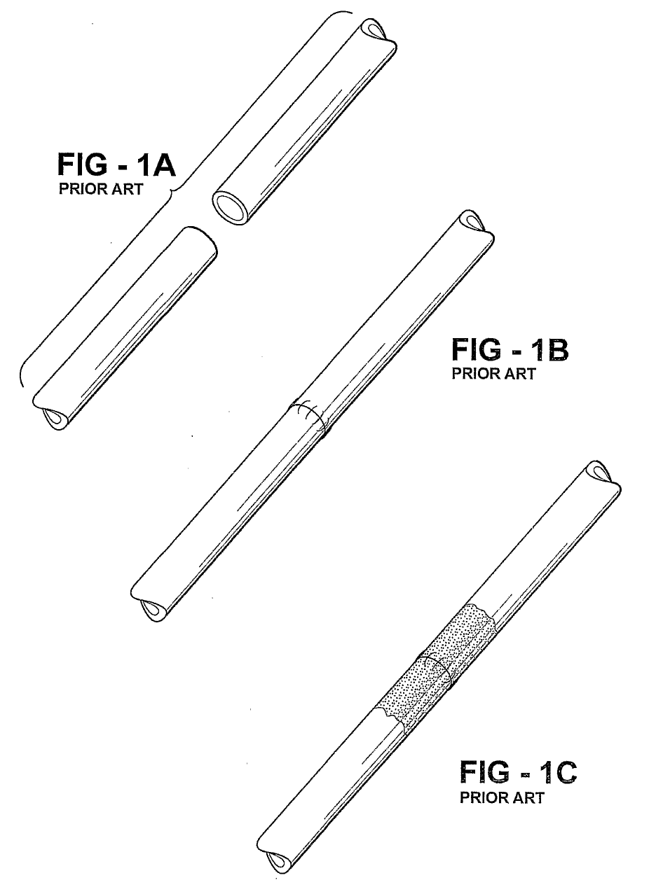

### DETAILED DESCRIPTION OF THE PREFERRED EMBODIMENTS

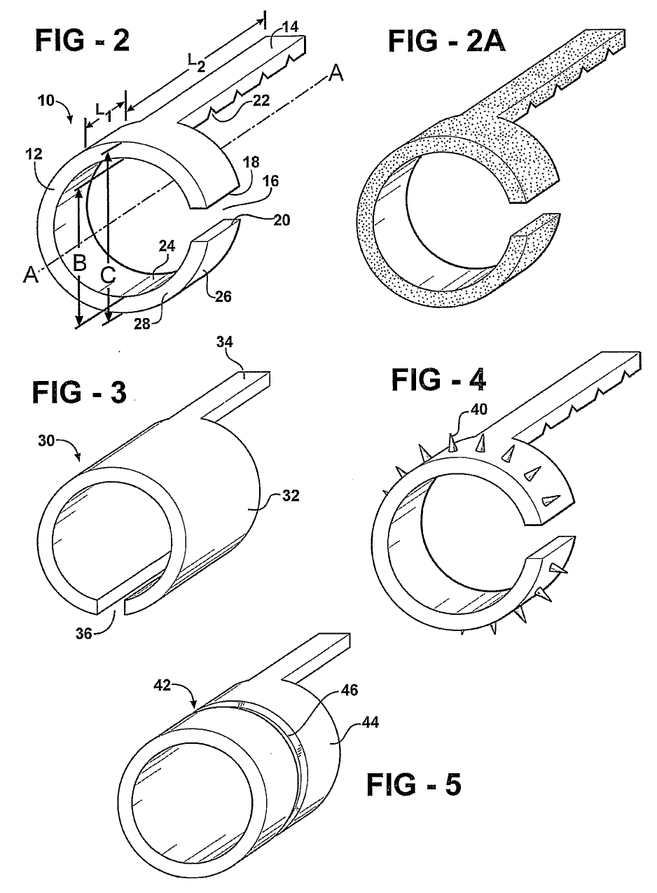

The present invention provides embodiments of apparatus and methods for performing anastomosis of hollow organs such as blood vessels. FIG. 2 illustrates a first embodiment of a blood vessel support 10 according to the present invention. The blood vessel support includes a vessel receiving portion 12 and a handle portion 14 extending therefrom. The vessel-receiving portion 12 may be said to be generally annular or formed as an annulus around an axis A-A. The vessel-receiving portion 12 may be generally cylindrical and extend along the axis A-A or may be shorter and more ringlike. It is preferred that the inner diameter B is generally constant along the longitudinal length L1 of the generally annular vessel-receiving portion 12. The vessel-receiving portion 12 may also be said to have an outer diameter C that is also preferably generally constant along the longitudinal length L1 of the vessel-receiving portion 12. As shown, the vessel-receiving portion 12 is an interrupted annulus rather than a complete annulus. As used herein, “interrupted annulus” means that the vessel-receiving portion 12 is generally annular with a cut or gap 16. The interruption of the annulus allows the vessel-receiving portion 12 to flex somewhat when a load is applied thereto. Described another way, the interrupted annulus forming the vessel-receiving portion 12 may be said to extend arcuately between a pair of spaced apart ends 18 and 20 with the space between the ends 18 and 20 defining the gap 16. The position, size and shape of the gap 16 may be different than illustrated in FIG. 2. The vessel-receiving portion may be said to have an inner surface 24, an opposed outer surface 26 and a leading edge 28.

The handle portion 14 may extend from the vessel-receiving portion 12 in a direction generally parallel to the axis A-A or may extend at an outward angle thereto. It is preferred that the handle portion 14 be removably interconnected with the vessel receiving portion 12 such that the handle portion 14 may be removed after an anastomosis procedure. In one approach, a frangible connection is provided between the handle portion and the vessel-receiving portion by defining a notch or weak point 22 at or near where the handle portion 14 joins the vessel-receiving portion 12. The handle portion 14 may be said to have an end-to-end length of L2.

FIG. 3 shows an alternative embodiment of a blood vessel support 30 according to the present invention. This embodiment differs from the embodiment of FIG. 2 in that the vessel-receiving portion 32 has a longer longitudinal length and the handle portion 34 is not removable. Also, the gap 36 in the interrupted annulus defining the vessel-receiving portion 32 is positioned in a different position relative to the handle portion 34.

The various dimensions of the vessel support according to the present invention may be varied from those illustrated in FIGS. 2 and 3. For microvessel (vessels with an outer diameter in the range of 0.3 to 4 mm) anastomosis, preferred embodiments of the present invention have an inner diameter in the range of 0.3 to 5 mm and a wall thickness equal or less than 0.5 mm. The vessel-receiving portion may have an end-to-end length L1 in the range of 2 to 10 mm with the length generally increasing with the diameter. The handle may have an end-to-end length L2 in the range of 10 to 30 mm with shorter lengths being preferred if the handle is not removable. The gap size and shape may vary, with typical distances between the ends 18 and 20 being in the range of 0.05 to 1 mm.

The vessel support according to the present invention may be provided with additional optional features. For example, as shown in FIG. 2A, the outer surface of the vessel receiving portion may be textured in order to assist in attachment between a blood vessel and the outer surface. Additionally or alternatively, as shown in FIG. 4, gripping elements 40 may be provided on the outer surface of the vessel receiving portion for gripping the adventitia of a blood vessel when the end of the vessel is everted about the outer surface of the vessel receiving portion, as will be described in more detail hereinbelow. The gripping elements 40 may take the form of small spikes or nubs that extend generally radially outwardly from the outer surface of the vessel receiving portion and may be positioned in a single row or staggered near the leading edge of the vessel receiving portion or may be provided across more of the surface or in multiple rows or multiple staggered rows. Alternatively, the gripping elements may be spaced from the front edge by a distance or may be positioned near the trailing edge. This can also include the presence of one or more continuous or non-continuous circular ridges or rings extending outwards on the outer surface 26 anywhere in between the ends of L1.

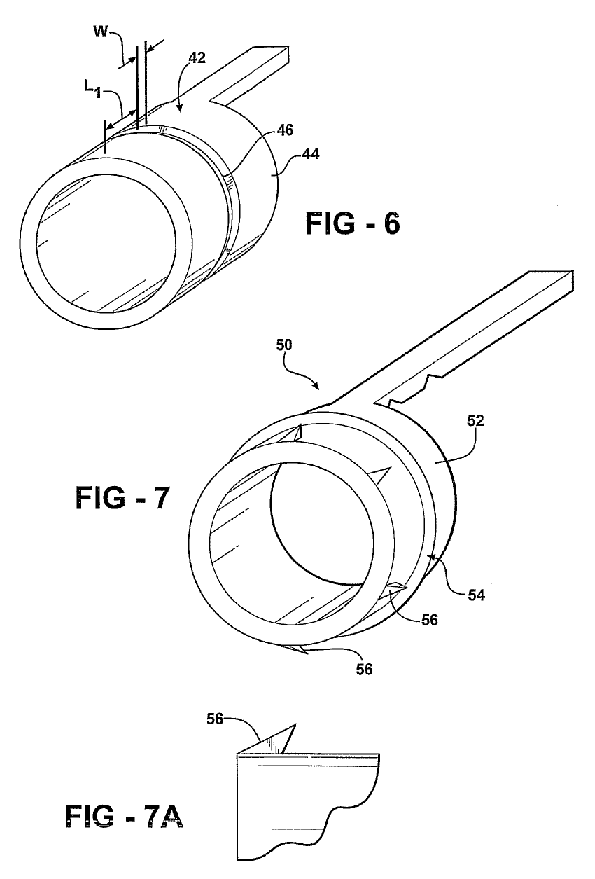

FIG. 5, illustrates a further embodiment of a vessel support 42. This embodiment differs from earlier embodiments in that the vessel-receiving portion 44 is annular but not interrupted. For all embodiments, interruption of the annular portion is optional, though preferred from most applications. This embodiment also includes a generally circumferential groove 46 in the outer surface of the vessel-receiving portion that may be used in the method, as will be described hereinbelow. The groove is an additional optional feature that may be used with any embodiment discussed herein. The “groove” may take other forms, such as being defined as a depression or recessed region between a pair of outwardly extending rings, bumps or gripping members. Referring to FIG. 6, the groove 46 may be spaced from the leading edge by a distance L1, which may be approximately half the length between the leading and trailing edges of the vessel receiving portion. The groove has a width W and a depth (not marked) that in some embodiments are both in the range of 250 to 750 microns. As will be clear to those of skill in the art, this width and depth should be sufficient to retain a suture when used in accordance with the method described hereinbelow. The groove may be semi-circular in cross section, triangular in cross section or may have other shapes.

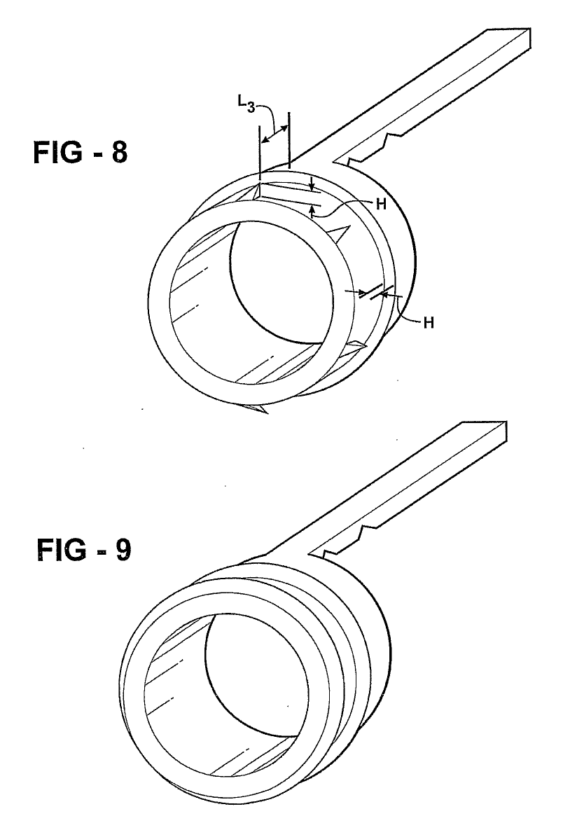

FIG. 7 illustrates another embodiment of a blood vessel support according to the present invention. As will be clear to those of skill in the art, in order to provide a groove 46 as shown in FIGS. 5 and 6, the wall thickness of the vessel receiving portion must be thick enough that the groove depth does not cause failure of the support. It is preferred that the wall thickness be as thin as possible while still allowing the support to function as intended. The embodiment of FIG. 7 allows the wall to be thinner while still providing a recessed region to receive a suture. The blood vessel support 50 in FIG. 7 has a vessel receiving portion 52 similar to previously described. The vessel receiving portion 52 has an outer surface with first and second raised elements defined thereon with the recessed region being defined between these raised elements. In this embodiment, one raised element 54 takes the form of a generally circumferential ring extending from the outer surface. The other raised element takes the form of a plurality of outwardly extending gripping elements 56 that are spaced apart circumferentially around the vessel receiving portion 52. In this embodiment, the gripping elements 56 are disposed adjacent the leading edge of the vessel receiving portion. FIG. 8 provides dimensions for this embodiment. The raised elements preferably each extend upwardly from the outer surface by a height h that is preferably approximately double the thickness of the wall of the vessel receiving portion. The thickness of the wall is preferably in the range of 50 to 400 microns, or as thin as will function properly. The two raised elements 54 and 56 are spaced apart by a distance L3 that may be in the range of 250 to 750 microns, which is sufficient for locating a suture in the generally circumferential recessed region defined between the elements 54 and 56.

The gripping elements 56 may take a variety of forms such as spikes, as shown in FIG. 4, nubs, hooks or barbed hooks. In the preferred and illustrated embodiment, the gripping elements 56 take the form of barbed hooks as best shown in the detail of FIG. 7a. The outer surface may be sloped at an angle of approximately 60 degrees with respect to a line extending perpendicularly from the outer surface. The rearward face of the barb may be undercut, as shown. The barb may also have an overall triangular shape, as illustrated, or may have other shapes.

FIG. 9 illustrates a further alternative wherein the ring of barbed hooks is replaced with a second ring.

The vessel support according to the present invention may be formed of a variety of materials. The preferred embodiments have at least the vessel-receiving portion formed of a resorbable and biocompatible material. As used herein a resorbable material means a material that is dissolved or otherwise broken down and assimilated or transported away by the body. Exemplary resorbable materials include hyaluronic acid in solid form or other biocompatible polymers. A vessel support according to the present invention may also be formed out of a non-resorbable material such as silicone, titanium or any other substance that is hard enough to resist the strain and is biocompatible. The handle portion is preferably unitarily formed with the remainder of the vessel support, but may be made of a different material or may be attached and/or detached in ways other than shown.

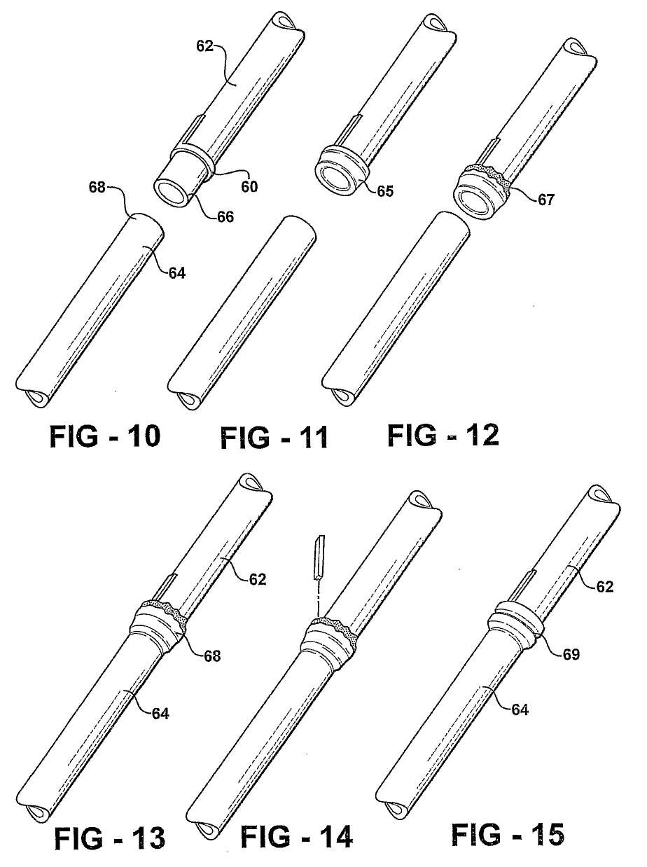

Referring now to FIGS. 10-14, a method for end-to-end vessel anastomosis according to an embodiment of the present invention will be described. According to the inventive method, a vessel support such as described above is provided. As shown in FIG. 10, this vessel support 60 is positioned around the end of a first vessel 62 such that the vessel 62 passes through the inner diameter of the vessel-receiving portion of the vessel support 60. The illustrated vessel support 60 corresponds to one of the earlier illustrated embodiments, but other versions may also be used. The inventive method provides for interconnecting the end of the first vessel 62 with the end of the second vessel 64. The first vessel may be said to have an end 66 while the second vessel may be said to have an end 68.

According to the inventive method, the end 66 of the first vessel 62 is everted around the outer diameter of the annulus of the vessel-receiving portion such that the inner surface 65 is directed outwardly, as shown in FIG. 7. A surgical adhesive 67 may then be applied to the everted end 66 of the first vessel as shown in FIG. 12. The adhesive may attach the everted end to the outer surface of the first vessel and/or to the vessel support 60. As shown in FIG. 13, the end 68 of the second vessel is then positioned over the everted end of the first vessel. This may require the end 68 of the second vessel 64 to be expanded, as shown. As shown in FIG. 13, a surgical adhesive is then applied to the location where the end of the second vessel 64 meets the outer surface of the first vessel 62. As will be clear to those of skill in the art, the joint formed by the inventive method of the present invention results in intima-to-intima contact of the two vessels that also provide structural support due to the overlapping of the two vessels and the use of the vessel support. As shown in FIG. 14, the handle portion of the vessel support may be removed. The removal of the handle may occur before or after the application of the adhesive as shown in FIG. 13.

In some versions of a method according to the present invention, the application of adhesive as shown in FIG. 12 may be skipped with the adhesive application as shown in FIG. 13 forming the joint.

Preferably, the adhesive used in the inventive method does not require the application of heat or radiant energy in order to activate and/or cure the adhesive. This avoids the need for additional surgical instruments for the application of heat or radiant energy. A preferred adhesive for the method of the present invention is a cyanoacrylate such as Omnex™, manufactured by Closure Medical and sold by Ethicon Products. Other adhesive means may be used if sufficiently effective, as will be clear to those of skill in the art.

Referring now to FIG. 15, a preferred alternative method of performing end-to-end vessel anastomosis will be described. This method is similar to the method shown in FIG. 10-14, but makes use of a vessel support as shown in FIGS. 5-9, which has a recessed region on the outer surface. This method starts with positioning the end of a vessel through the vessel receiving portion and everting the end, as shown in FIGS. 10 and 11. The end of the second vessel is then positioned over the everted end of the first vessel and secured in place with the suture 69. The suture is generally aligned with and cooperates with the recessed region, such as groove 46 or the region between raised elements 54 and 56 on the outer surface of the vessel receiving portion to secure the vessel ends to each other. The approach avoids placing the suture in contact with blood flow in the vessel and maintains intima-to-intima contact. As shown in FIG. 15, the suture 60 may be said to extend generally circumferentially around the overlapped ends of the vessels. Alternatively, the suture may be used with adhesive. For example, the everted end of the first vessel may be adhesively attached in place as shown in FIG. 12, with the end of the second vessel being secured into place using the suture. Alternatively, adhesive may secure the end of the second vessel in place as shown in FIG. 13 with the suture serving as a reinforcement or support. As mentioned previously, the annular portion may be non-interrupted in this embodiment, which provides a more stable support when the suture is tightened. Alternatively, an interrupted annulus may be used. The groove feature or recessed region preferably has an effective depth of at least 100 microns and may be provided on any embodiment of vessel support described herein and in combination with any of the other optional features.

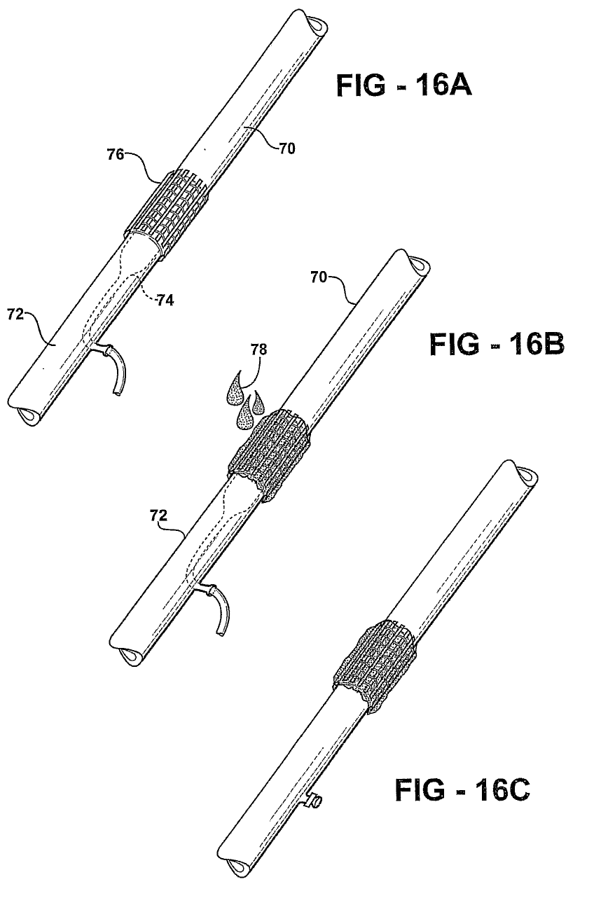

Referring now to FIGS. 16A-16C, an alternative method for performing end-to-end vessel anastomosis is illustrated. The method illustrated in FIGS. 16A-16C is preferably for larger vessels, such as vessels with an outer diameter greater than 4 mm. In this method, a pair of vessels 70 and 72 are butted end to end with an intraluminal support, such as an expandable stent 74, positioned so as to support the butted joint. A meshlike patch or wrap 76 is then positioned so as to span the joint and an adhesive 78 such as discussed above is applied to the joint and the mesh 76. The mesh may be formed of a resorbable or non-resorbable material. The mesh preferably allows some expansion and contraction of the vessel so as to allow the passage of pressure waves. The support is then removed as shown in FIG. 16C.

As will be clear to those of skill in the art, the illustrated and discussed embodiments of the present invention may be altered in various ways without departing from the scope or teaching of the present invention. It is the following claims, including all equivalents, which define the scope of the present invention.

1. A method for performing end to end vessel anastomosis comprising:
providing a vessel support comprising a vessel receiving portion and a handle portion extending therefrom, the vessel receiving portion comprising an annulus having an inner diameter and an outer diameter;
positioning the vessel receiving portion of the vessel support around the end of a first vessel such that the end of the first vessel is disposed thru the inner diameter of the vessel receiving portion;
everting the end of the first vessel around the outer diameter of the annulus of the vessel receiving portion such that an inner surface of the vessel is directed outwardly;
positioning the end of a second vessel over the everted end of the first vessel such that an inner surface of the second vessel is disposed against the inner surface of the first vessel;
providing a surgical adhesive; and
adhering the end of the vessels together using the surgical adhesive, the adhering not requiring the application of heat or radiant energy.

2. The method of claim 1, wherein the annular vessel receiving portion is interrupted so as to define a pair of spaced apart ends with a gap therebetween.

3. The method of claim 1, further comprising adhering the everted end of the first vessel to the vessel support or to the vessel prior to positioning the end of the second vessel over the everted end of the first vessel.

4. The method of claim 1, wherein the handle portion is interconnected with the vessel receiving portion by frangible connection, the method further comprising removing the handle portion from the vessel receiving portion.

5. The method of claim 1, wherein the vessel receiving portion is formed of a resorbable material.

6. The method of claim 1, wherein the vessel receiving portion has a generally circumferential groove formed in an outer surface thereof.

7. The method of claim 6, further comprising:
positioning a suture around an outer surface of the end of the second vessel after the end of the second vessel is positioned over the everted end of the first vessel, the suture being generally aligned with the groove in the outer surface of the vessel receiving portion.

8. The method of claim 1, wherein the vessel receiving portion has a plurality of gripping elements extending outwardly from an outer surface thereof.

9. A method for performing end to end vessel anastomosis comprising:
providing a vessel support comprising a vessel receiving portion and a handle portion extending therefrom, the vessel receiving portion being generally annular and having an inner diameter and an outer diameter, the generally annular vessel receiving portion having an outer surface with a grove defined therein;
positioning the vessel receiving portion of the vessel support around the end of a first vessel such that the end of the first vessel is disposed thru the inner diameter of the vessel receiving portion;
everting the end of the first vessel around the outer diameter of the annulus of the vessel receiving portion such that an inner surface of the vessel is directed outwardly;
positioning the end of a second vessel over the everted end of the first vessel such that an inner surface of the second vessel is disposed against the inner surface of the first vessel; and
positioning a suture around an outer surface of the end of the second vessel after the end of the second vessel is positioned over the everted end of the first vessel, the suture being generally aligned with the groove in the outer surface of the vessel receiving portion.

10. The method of claim 9, further comprising:
providing a surgical adhesive; and
adhering the end of the second vessel to the everted end of the first vessel together using the surgical adhesive, the adhering not requiring the application of heat or radiant energy.

11. A method for performing end to end vessel anastomosis comprising:
providing a vessel support comprising a vessel receiving portion and a handle portion extending therefrom, the vessel receiving portion being generally annular and having an inner diameter and an outer diameter, the generally annular vessel receiving portion having an outer surface with a recessed region defined thereon;
positioning the vessel receiving portion of the vessel support around the end of a first vessel such that the end of the first vessel is disposed thru the inner diameter of the vessel receiving portion;
everting the end of the first vessel around the outer diameter of the annulus of the vessel receiving portion such that an inner surface of the vessel is directed outwardly;
positioning the end of a second vessel over the everted end of the first vessel such that an inner surface of the second vessel is disposed against the inner surface of the first vessel; and
positioning a suture around an outer surface of the end of the second vessel after the end of the second vessel is positioned over the everted end of the first vessel, the suture being generally aligned with the recessed region on the outer surface of the vessel receiving portion.

12. The method of claim 11, wherein:
the outer surface of the vessel receiving portion have first and second raised elements defined thereon and extending upwardly from the outer surface, the recessed region being defined as the region between the raised elements.

13. The method of claim 12, wherein:
at least one of the raised elements is a circumferential ring.

14. The method of claim 12, wherein:
at least one of the raised elements comprises a plurality of gripping elements extending upwardly from the outer surface of the vessel receiving portion, the gripping elements being spaced apart circumferentially around the vessel receiving portion.

15. A blood vessel support for performing an end to end vessel anastomosis, the blood vessel support comprising:
a vessel receiving portion formed of a biocompatible material, the vessel receiving portion having a generally annular shape with an outer surface and an inner surface, the inner surface defining an inner diameter of the vessel receiving portion, the vessel receiving portion having a wall thickness defined between the inner and outer surfaces; and
a handle portion interconnected with the vessel receiving portion and extending therefrom.

16. The blood vessel support of claim 15, wherein the generally annular vessel receiving portion is interrupted so as to define a pair of spaced apart ends with a gap therebetween.

17. The blood vessel support of claim 15, further comprising a groove defined in the outer surface of the generally annular vessel receiving portion.

18. The blood vessel support of claim 15, wherein the inner diameter of the vessel receiving portion is in the range of 0.3 to 5 mm and the wall thickness of the vessel receiving portion is less than or equal to 0.5 mm.

19. The blood vessel support of claim 15, wherein the vessel receiving portion is formed of a resorbable material.

20. The blood vessel support of claim 15, wherein the handle portion is interconnected with the vessel receiving portion by frangible connection.

21. A blood vessel support for performing an end to end vessel anastomosis, the blood vessel support comprising:
a vessel receiving portion formed of a biocompatible material, the vessel receiving portion having a generally annular shape with an outer surface and an inner surface, the inner surface defining an inner diameter of the vessel receiving portion, the vessel receiving portion having a wall thickness defined between the inner and outer surfaces, the outer surface having a first raised element and a second raised element defined thereon, each raised element extending upwardly from the outer surface and also extending generally circumferentially around the vessel receiving portion, a generally circumferential recessed region being defined between the first and second raised elements; and
a handle portion interconnected with the vessel receiving portion and extending therefrom.

22. The blood vessel support of claim 21, wherein:
at least one of the raised elements is a circumferential ring.

23. The blood vessel support of claim 21, wherein:
both of the raised elements are circumferential rings.

24. The blood vessel support of claim 21, wherein:
at least one of the raised elements comprises a plurality of gripping elements extending upwardly from the outer surface of the vessel receiving portion, the gripping elements being spaced apart circumferentially around the vessel receiving portion.

25. The blood vessel support of claim 24, wherein:
the vessel receiving portion has a leading edge and a trailing edge;
the gripping elements being disposed adjacent the leading edge.

26. The blood vessel support of claim 24, wherein the gripping elements comprise spikes or hooks extending generally radially outwardly from the outer surface.

27. The blood vessel support of claim 21, wherein the generally annular vessel receiving portion is interrupted so as to define a pair of spaced apart ends with a gap therebetween.

28. The blood vessel support of claim 21, wherein the inner diameter of the vessel receiving portion is in the range of 0.3 to 5 mm and the wall thickness of the vessel receiving portion is in the range of 50 to 400 microns.

29. The blood vessel support of claim 21, wherein the vessel receiving portion is formed of a resorbable material.

30. The blood vessel support of claim 21, wherein the handle portion is interconnected with the vessel receiving portion by frangible connection.
